(19) United States
(12) Patent Application Publication
(10) Pub. No.: US 2021/0106748 A1
(43) Pub. Date: Apr. 15, 2021

(54) MULTI-LAYER ASPIRATION TUBING DESIGN FOR REDUCED POST OCCLUSION SURGE AND PUMP PULSATION

(71) Applicant: Johnson & Johnson Surgical Vision, Inc., Santa Ana, CA (US)

(72) Inventor: Chetan Nirkhe, Aliso Viejo, CA (US)

(21) Appl. No.: 17/064,347

(22) Filed: Oct. 6, 2020

Related U.S. Application Data
(60) Provisional application No. 62/913,634, filed on Oct. 10, 2019.

(51) Int. Cl. A61M 3/02; A61F 9/007; A61M 1/00

(52) U.S. Cl. CPC A61M 3/0283; A61F 9/00745; A61M 1/008; A61M 2205/3331; A61M 2205/52; A61M 2205/505; A61M 2205/12; A61M 1/0076

(57) ABSTRACT

Multilayer tubing balances the property of improved kink resistance, reduces post occlusion surge, and reduces pump pulsations after surge. A multilayer construction is provided for tubing for phacoemulsification systems. Multilayer tubing includes multiple layers having an inner layer and an outer layer, wherein the material of the inner layer is harder or stiffer than the material of the outer layer.

### CROSS-REFERENCE TO RELATED APPLICATIONS

This application claims the benefit under 35 U.S.C. § 119(e) of U.S. Provisional Patent Application No. 62/913,634 filed Oct. 10, 2019, which is incorporated herein by reference in its entirety.

### BACKGROUND OF THE INVENTION

### Field of Technology

The present invention relates generally to the reduction of post occlusion surge and pump pulsation and, more particularly, multilayer aspiration tubing balancing the property of improved kink resistance and reducing post occlusion surge and pump pulsations.

### Description of the Background

During ophthalmic surgery, an ophthalmic surgical apparatus is used to perform surgical procedures in a patient's eye. An ophthalmic surgical apparatus typically includes a handheld medical implement or tool, such as a hand piece with a tip and/or sleeve, and operating controls for regulating settings or functions of the apparatus and tool. Operation of the tool requires control of various operating settings or functions based on the type of tool used. Such apparatuses typically include a control module, power supply, an irrigation source, one or more aspiration pumps, as well as associated electronic hardware and software for operating a multifunction handheld surgical tool. The hand piece may include a needle or tip which is ultrasonically driven once placed with the incision to, for example, emulsify the lens of the eye. In various surgical procedures, these components work together in order to, for example, emulsify eye tissue, irrigate the eye with a saline solution, and aspirate the emulsified lens from the eye.

Intraocular pressure (TOP) is the fluid pressure inside the anterior chamber of the eye. In a normal eye, intraocular pressure may vary depending on the time of day, activities of the patient, fluid intake, medications, etc. Intraocular pressure may be measured as static (a specific value) or dynamic (a range of values). As can be appreciated, the static IOP and dynamic IOP of a patient's eye can fluctuate greatly during an ophthalmic surgery procedure. It is well known that the IOP in an anterior chamber of the eye is required to be controlled and maintained during such surgical procedures in order to avoid damage to the patient's eye. For the correct function of the eye and its structure (e.g. shape) and to preserve sharp and undamaged vision, it is very important to keep the IOP in normal, physiological values.

An exemplary type of ophthalmic surgery is phacoemulsification. Phacoemulsification includes making a corneal and/or scleral incision and the insertion of a phacoemulsification hand piece that includes a needle or tip that is ultrasonically driven to emulsify, or liquefy, the lens. A phacoemulsification system typically includes a hand piece coupled to an irrigation source and an aspiration pump. The hand piece includes a distal tip that emits ultrasonic energy to emulsify a crystalline lens within the patient's eye. The hand piece includes one or more irrigation ports proximal to the distal tip and coupled to the irrigation source via an irrigation input line. The hand piece further includes an aspiration port at the distal tip that is coupled to the aspiration pump via an aspiration output line. Concomitantly with the emulsification, fluid from the irrigation source (which may be a bottle or bag of saline solution that is elevated above the patient's eye, to establish positive pressure by gravity, and/or with external pressure source) is irrigated into the eye via the irrigation line and the irrigation port(s). This fluid is directed to the crystalline lens in the patient's eye in order to maintain the anterior chamber and capsular bag and replenish the fluid aspirated away with the emulsified crystalline lens material. The irrigation fluid in the patient's eye and the crystalline lens material is aspirated or removed from the eye by the aspiration pump and line via the aspiration port.

Similarly, cataract surgery is a complex procedure performed by highly skilled surgeons using extremely complex and expensive equipment. The surgeon undergoes years of training to perfect their technique while using only a fraction of the system's capabilities and features. For example, cataract tissue, which may be of varying densities, and may be removed by aspiration during the phacoemulsification procedure. During phacoemulsification, the natural lens is broken up into pieces and brought to the tip of the hand piece for removal. However, pieces of lens material may occlude or block the tip of the needle until the material is emulsified into smaller pieces. When an occlusion of the hand piece tip occurs, vacuum builds up in the hand piece and tubing lines. This build up of vacuum can result in an influx of fluid and material to be aspirated out of the eye at a high rate resulting in an imbalance in the anterior chamber called post occlusion surge (POS). Post occlusion surge could potentially cause damage to the structures of the eye. In practice, the surgeon may anticipate this occurrence and discontinue ultrasonic power and irrigation/aspiration to prevent any damage to the eye. If the occlusion break occurs faster than the surgeon can discontinue power and vacuum, the surgeon will not be able to prevent the post occlusion surge. Studies have shown that the human reaction time is approximately 350 milliseconds (ms). That means that the patient may be subjected to an additional 350 ms or more of ultrasonic energy and vacuum every occlusion break.

During the phacoemulsification process for cataract removal, a disposable plastic cassette pack with a drain bag is typically used to collect effluent material. The cassette pack typically consists of a tubing cassette which has one or more flow paths for fluid. Further, the cassette pack typically consists of one or more valves to control fluid flow. The cassette pack typically has attached tubing which moves fluid to and from the eye through a surgical hand piece.

Current aspiration tubing on ophthalmic cassettes typically use a single layer extruded tubing which is susceptible to kinks during handling at time of surgery. For example, the OP073 Pack used with the WHITESTAR SIGNATURE PRO phacoemulsification system uses aspiration tubing having a single layer of material having a nominal wall thickness (e.g. the material may be a polyvinyl chloride (PVC) with a Shore 70A and having a nominal wall thickness of about 0.048″). Relatively thick tubing, which may reduce compliance in the tubing, adds to the weight of a long aspiration tube, such as an aspiration tube that is six feet long. This type of aspiration tube is somewhat difficult to maneuver when attached to a phacoemulsification (phaco) or Irrigation/Aspiration (IA) hand piece. Further, this type of aspiration tube is prone to kinks during normal handling.

Other approaches utilized by the prior art include small inner diameter (ID) thick walled plasticized PVC aspiration tubing or similar material that can still be relatively flexible enough for packaging. The small ID may help reduce pump pulsation occurring due to the inherent nature of peristaltic pump rollers moving over tubing method, but at a cost of higher free running vacuum. Thicker tubing of the prior art adds weight to the overall tubing and may make it difficult to maneuver when attached to the hand piece during surgery. A single layer tubing construction with stiff material tubing is bulky, difficult to handle, and difficult to coil and package without kinks. Once aspiration tubing is kinked, fluid flow through the tubing may become obstructed. Further, a dampening effect of pump pulsations may be attributed to single wall tubing, depending on the ID of the tubing. In some instances, the smaller the ID, the lower the pump pulsations regardless of flow rate or vacuum. Yet, even further, smaller tubing may be prone to lens particle clogging.

U.S. Pat. No. 7,981,074 B2, issued to Davis et al. (“the Davis system”), describes a surgical system with irrigation and aspiration lines. The irrigation and aspiration lines of the Davis system have different compliance or stiffness. For example, the irrigation line may have a higher compliance than the aspiration line. The Davis system describes reducing the compliance in an aspiration line by increasing a wall thickness relative to an irrigation line. Also described is an aspiration line being made from a material having a relatively higher stiffness, or durometer.

U.S. Pat. No. 8,257,298, issued to Hamboly (“the Hamboly multi-tube”) describes a multi-tube catheter. The Hamboly multi-tube includes an assembly of two or more tubes fused together to form one catheter tube shaft. Each tube includes at least one lumen extending longitudinally through the catheter from its distal end to its proximal end. The two or more tubes are fused together through the application of heat and pressure. The fused tubes form a single catheter tube that can separate at each end, thereby creating split ends.

U.S. Patent App. Pub. No. 2009/0205736 A1, filed by Mezzalira (“the Mezzalira hose”), describes a flexible hose, particularly for transporting food liquids. The Mezzalira hose comprises at least one outer protective layer of a first flexible polymer material and at least one inner layer of a second flexible polymer material. The second polymer material comprises a thermoplastic compound of polyolefin nature and an elastomer compound in such a weight percentage as to impart a Shore A hardness of less than 85. The second polymer material is selected from polymer materials substantially free of liquid plasticizer agents.

U.S. Patent App. Pub. No. 2011/0172644 A1, filed by Zanoni et al. (“the Zanoni shaft”), describes a central venous catheter having an outer tubular member and an inner tubular member that are formed as a single integrated tube containing material of different durometer and varying amounts of radiopaque filler material. The Zanoni shaft describes wherein the polymer durometer of the inner tubular member is higher than the polymer durometer of the outer tubular member. The combination of the higher durometer inner tubular member and the lower durometer outer tubular member along the length of the tube provides the desired tensile strength, hardness, chemical resistance, and fatigue resistance.

There is a need in the art for an aspiration tube construction and a need for making such an aspiration tube construction which can provide advantages over the above-mentioned fluid tubing which can prevent tubing kinks during surgical procedures, such as cataract surgical procedures. The aspiration of a cataract lens happens using a peristaltic pump. In many cases, the pump pulsations influence intra-ocular pressure in the patient's eye. Any remediation to help reduce post occlusion surge and pump pulsations will assist in a stable eye chamber during a surgical operation.

### SUMMARY OF THE INVENTION

The present embodiments may relate to, inter alia, systems and methods for reducing post occlusion surge and pump pulsation. Some embodiments of the present disclosure may use, for example, a multilayer construction wherein the inner layer of a tubing construction comprises a rigid or stiff material and an outer layer of the tubing construction comprises of relatively soft material.

A composition of a multi-layer aspiration tubing may improve kink resistance and reduce pump pulsation with sufficient flexibility for handling and packaging of the tubing. The multi-layer tubing may comprise of a stiff inner layer and a softer outer layer. A stiff inner layer may improve kink resistance and reduce compliance thus reducing post occlusion surge and pump pulsations. A softer outer layer would keep the tubing flexible and provide easy handling and allow for coiling of the tubing without kinks. Further, a smaller overall wall thickness due to the advantage of a stiff inner layer may reduce the overall weight of the tubing.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the systems and methods disclosed therein. It should be understood that each figure depicts an embodiment of a particular aspect of the disclosed systems and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present embodiments are not limited to the precise arrangements and are instrumentalities shown, wherein:

FIG. 1 schematically illustrates an eye treatment system in which a cassette couples an eye treatment probe with an eye treatment console;

FIG. 2A illustrates a cross-sectional view of a two-layer tubing construction in accordance with at least one embodiment;

FIG. 2B illustrates a side-sectional view of a two-layer tubing construction in accordance with at least one embodiment;

FIG. 3 illustrates a test matrix of a two-layer tubing construction in accordance with at least one embodiment;

FIG. 4 illustrates a delta IOP—surge response for different tubing types test matrix of a two-layer tubing construction in accordance with at least one embodiment;

FIG. 5 illustrates a POS response comparison for different aspiration tubing types for two-layer tubing construction and reduction in pulsation after the POS in accordance with at least one embodiment; and

FIG. 6 illustrates a test set-up for measuring flexibility of plastic tubing.

The figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical surgical, and particularly optical surgical, apparatuses, systems, and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to the disclosed elements and methods known to those skilled in the art.

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Embodiments are provided throughout so that this disclosure is sufficiently thorough and fully conveys the scope of the disclosed embodiments to those who are skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that exemplary embodiments may be embodied in different forms. As such, the exemplary embodiments should not be construed to limit the scope of the disclosure. As referenced above, in some exemplary embodiments, well-known processes, well-known device structures, and well-known technologies may not be described in detail.

A surgical cassette, also referred to as a medical pack, a fluidics cassette, or simply, a cassette, is typically used to facilitate irrigation and aspiration during surgical procedures, such as phacoemulsification surgery. The surgical cassette may be inserted and mounted to a surgical console and become part of an overall phacoemulsification surgery system. The surgical cassette may perform a myriad of functions, such as effluent material collection, vacuum, pressure, and/or fluid flow sensing, and the control of fluid flow through tubing encased within the cassette and between a surgical handpiece and a surgical console.

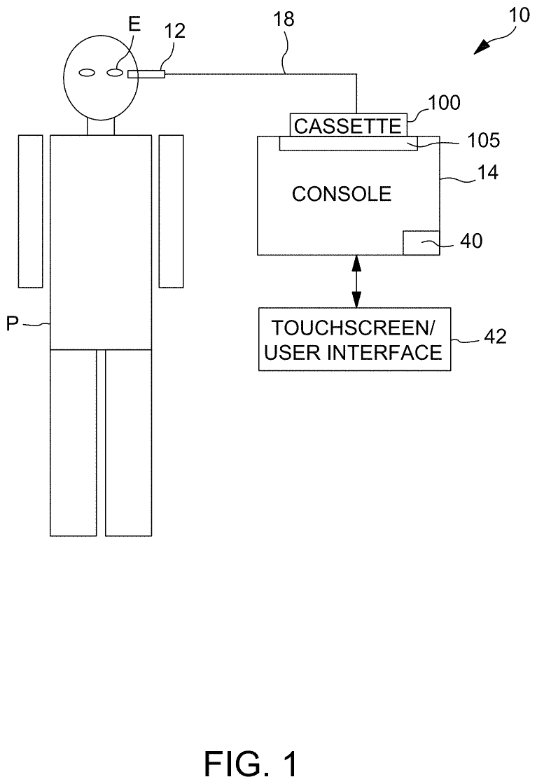

Referring to FIG. 1, a system 10 for treating an eye E of a patient P generally includes an eye treatment probe hand piece 12 coupled to a console 14 by a cassette 100 mounted on the console via interface 105. Hand piece 12 may include a handle for manually manipulating probe tip inserted to an eye. The probe tip has a distal end with one or more lumens in the probe tip allowing irrigation fluid to flow from the console 14 and/or cassette 100 into the eye. Aspiration fluid may also be withdrawn through a lumen of the probe tip, with the console 14 and cassette 100 generally including a vacuum aspiration source, a positive displacement aspiration pump, or both to help withdraw and control a flow of surgical fluids into and out of eye E. As the surgical fluids may include biological materials that should not be transferred between patients, cassette 100 will often be disposable or comprise a disposable structure or structure capable of being sterilized for re-use, with the surgical fluids being transmitted through conduits of the cassette that avoid direct contact in between those fluids and the components of console 14.

When a distal end of the probe tip of hand piece 12 is inserted into an eye E, for example, for removal of a lens of a patient with cataracts, an electrical conductor and/or pneumatic line (not shown) may supply energy from console 14 to an ultrasound transmitter of the hand piece, a cutter mechanism, or the like. Alternatively, the hand piece 12 may be configured as an irrigation/aspiration (I/A) or vitrectomy hand piece. Also, the ultrasonic transmitter may be replaced by other means for emulsifying a lens, such as a high energy laser beam. The ultrasound energy from hand piece 12 helps to fragment the tissue of the lens, which can then be drawn into a port of the tip by aspiration flow. To balance the volume of material removed by the aspiration flow, an irrigation flow through hand piece 12 (or a separate probe structure) may also be provided, with both the aspiration and irrigations flows being controlled by console 14.

To avoid cross-contamination between patients and/or to avoid incurring excessive expenditures for each procedure, cassette 100 and its one or more conduits (e.g an irrigation line and an aspiration line) illustrated by tubing line 18 may be disposable. Alternatively, the conduit or tubing may be disposable, with the cassette body and/or other structures of the cassette being reusable following sterilization. Regardless, the disposable components of the cassette are typically configured for use with a single patient and may not be suitable for sterilization. In some embodiments, the cassette may interface with reusable (and often quite expensive) components of console 14, which may include one or more peristaltic pump rollers, a Venturi or other vacuum source, a controller 40, or the like.

Controller 40 may include an embedded microcontroller and/or many of the components common to a personal computer, such as a processor, data bus, a memory, input and/or output devices (including a touch screen user interface 42), and the like. Controller 40 may include both hardware and software, with the software typically comprising machine readable code or programming instructions for implementing one, some, or all of the methods described herein. The code may be embodied by a tangible media such as a memory, a magnetic recording media, an optical recording media, or the like. Controller 40 may have (or be coupled to) a recording media reader or the code may be transmitted to controller 40 by a network connection such as an internet, an intranet, an Ethernet, a wireless network, or the like. Along with programming code, controller 40 may include stored data for implementing the methods described herein and may generate and/or store data that records parameters corresponding to the treatment of one or more patients. Many components of console 14 may be found in or modified from known commercial phacoemulsification.

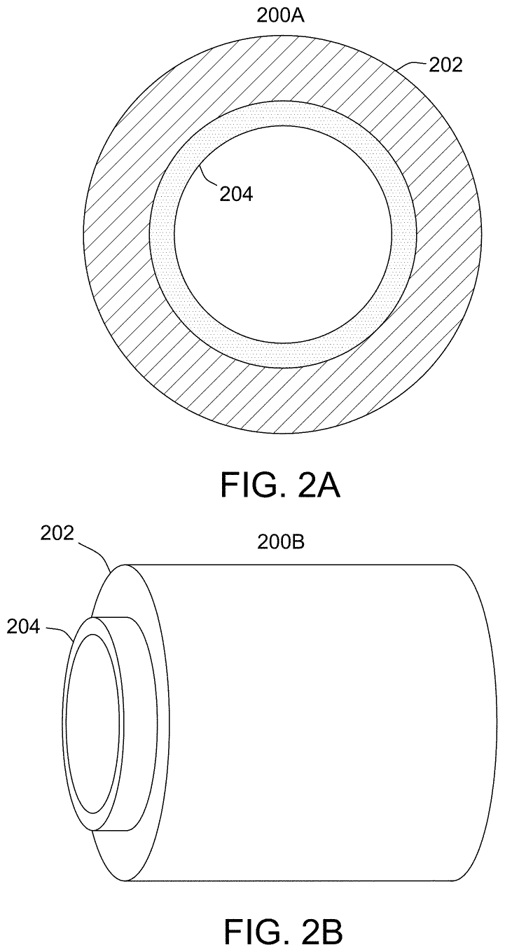

FIG. 2A illustrates a cross-sectional view 200A of a two-layer tubing construction in accordance with the disclosed invention. In the illustrated example, the multi-layer tubing includes two layers, however it is to be understood that more, or less, than two layers may be implemented. In at least one embodiment, the inner layer 204 may be comprised of one or more rigid or stiff materials or one or more materials stiffer than outer layer 202. As known in the art, lower Shore A values indicate soft materials and higher Shore A values indicate more rigid or stiff materials. An example of a rigid or stiff material may be a nylon material of a certain thickness and hardness. In an embodiment, the rigid or still material may be plasticized PVC, Polyolefins, Nylons (e.g. Nylon6, Nylon 66, Nylon 12, etc.) and/or Pebax of a higher Shore A value. In another embodiment, the rigid or stiff material may comprise braided tubing (with different braid materials such as stainless steel) which provides stiffness and kink resistance. In an embodiment, the inner layer 204 may have a thickness from 0.001″ to 0.10″, preferably from 0.005″ to 0.015″.

The outer layer 202 may be comprised of one or more softer materials, when compared to materials used for the inner layer. For example, the outer layer may be comprised of one or more Pebax materials and be of a certain thickness. In an embodiment, the softer material may be plasticized PVC grades less than or equal to a Shore A value of 65, or silicone or Pebax with a similar Shore A value. In an embodiment, the outer layer 202 may have a thickness from 0.002″ to 0.080″, preferably about 0.015″ to about 0.050″. In an embodiment, when two or more different materials with different Shore A values are used they may be chosen from the same family of materials, e.g. different grades of PVC, Pebax, or Nylon. In an embodiment, if dissimilar materials are used, typically a tie layer is used to bond the materials during processing.

According to an embodiment, it is envisioned that if the outer layer 202 has a lower Shore A value than the inner layer 204 then the inner layer 204 may be considered rigid or stiff. In an embodiment, 65A and lower shores of plasticized PVC may be considered soft and 65A and above may be considered rigid or stiff for plasticized PVC.

The advantage of a stiff inner layer may be the improvement of kink resistance of the tubing, such as tubing 18 of FIG. 1 between hand piece 12 and console 14. A stiff inner layer of the tubing may reduce compliance. Even further, a stiff inner layer may reduce post occlusion surge and pump pulsations.

The advantage of a soft outer layer of the tubing, such as tubing 18 of FIG. 1 between hand piece 12 and console 14 is the soft outer layer of the tubing may allow increased flexibility of tubing 18. In some embodiments, the soft outer layer may allow for easy handling of tubing 18. Even further, a soft outer layer of tubing may allow for coiling of tubing 18 without any kinks.

Overall, the two-layer example construction of the disclosed invention may have a smaller overall wall thickness due to the inclusion of a stiff inner layer 204. Due to a smaller thickness, the overall weight of the tubing 18 may be less in comparison with a single layer bulky tubing construction. Further, it should be noted that the views provided by the figures are not to scale and are merely shown for illustrative purposes.

FIG. 2B illustrates a side-sectional view 200B of a two-layer tubing construction in accordance with the disclosed invention. In the illustrated example, as set forth above with respect to FIG. 2A, the multi-layer tubing includes two layers, however it is to be understood that more, or less, than two layers may be implemented. In at least one embodiment, the inner layer 204 may comprise of stiff materials. An example of a stiff material may be a nylon material of a certain thickness. The outer layer 202 may comprise of softer materials, when compared to materials used for the inner layer. For example, the outer layer may comprise of Pebax materials of a certain thickness. As described above, the two-layer construction may allow for smaller overall wall thickness, improved handling, and reduced post occlusion surge and pump pulsations.

The multi-layer tubing design illustrated may be produced using a coextrusion technique common in the plastics processing industry. The construction may also help the socket bonding capability of the tubing due to its soft outer layer which will conform better to the geometry of the fitting compared to a single layer hard tubing material.

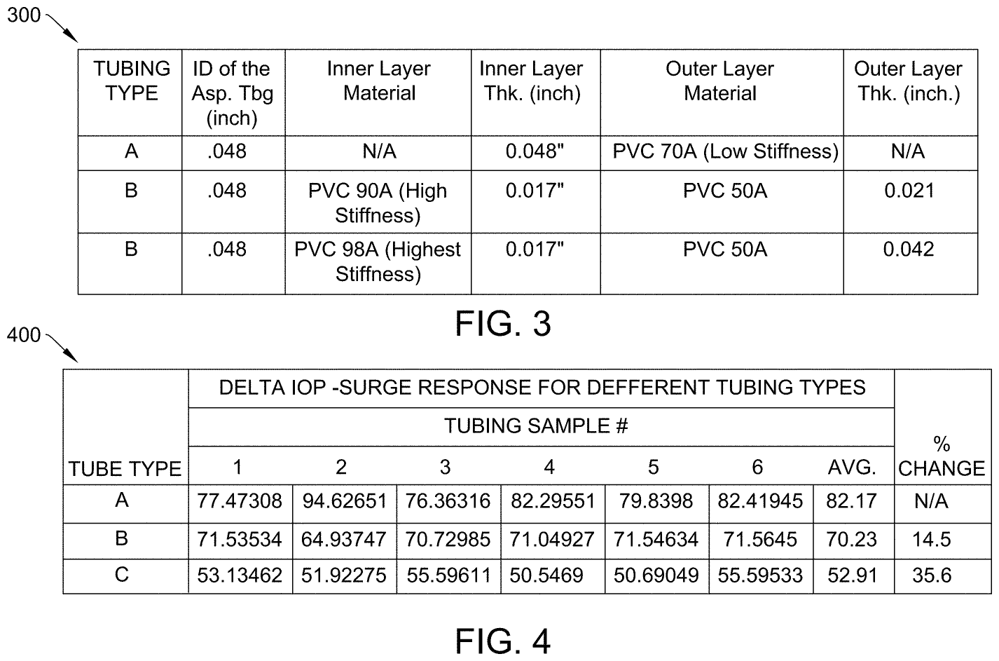

FIG. 3 illustrates a test matrix 300 of a two-layer tubing construction in accordance with at least one embodiment of the disclosed invention. In view of matrix 300, a total of six samples each of three types of aspiration tubing were tested. In the example, a stiff inner layer thickness was tested by varying the durometer of PVC tubing. In this example, occlusion surge response was tested on a WHITESTAR SIGNATURE PRO system with A, B, and C tubing samples inside a rigid eye chamber model with an attached pressure sensor. The occlusion was simulated for approximately three seconds each. Data was collected for IOP (mmHg) and time (milliseconds) using Data Acquisition System via LabVIEW software from National Instruments. For data analysis, an average change in IOP (Delta IOP) for occlusion surge was determined.

FIG. 4 illustrates a result summary 400 of test matrix 300. Result summary 400 shows the delta IOP surge response for the different tubing types of test matrix 300 of a two-layer tubing construction in accordance with at least one embodiment of the disclosed invention. In this illustrative example, the lower the delta IOP, the better the surge response. In the provided example, a single wall/material tubing produced the highest Delta IOP. The stiffest inner layer tubing type, group C, reduced the surge by 36 percent. The stiffer material with reduced compliance helped reduce pressure drop as the stiffer tubing wall helped dampen surge.

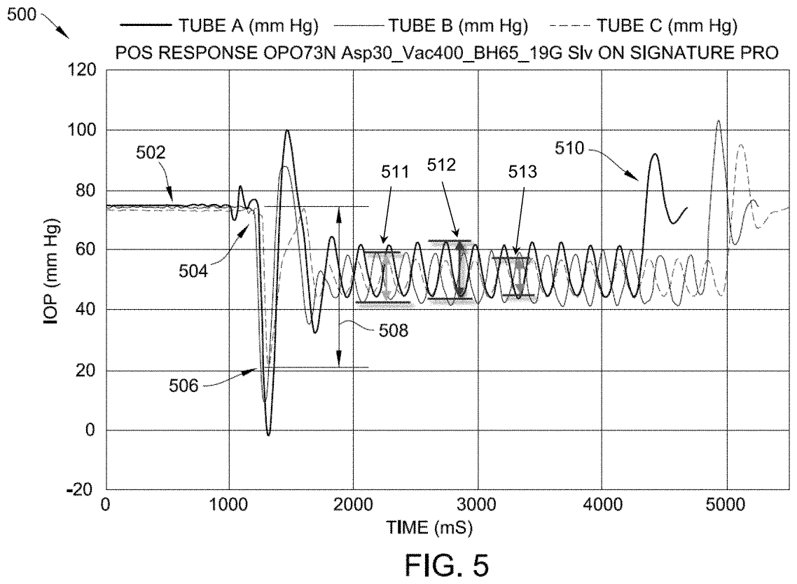

FIG. 5 shows graph 500 that illustrates a POS response comparison for different types of tubing constructions, including two-layer tubing constructions in accordance with at least one embodiment of the disclosed invention. As shown, part 502 of the graph indicates steady state bottle pressure. At step 504, the aspiration tubing is occluded. At step 506, the occlusion has been released. At step 508, Delta IOP can be measured in mmHg. At step 510, the foot pedal of the surgical system is released. It can be observed that not only is the Delta IOP reduced with a stiffer inner layer but also overall peak to trough. The pulsation after the surge is also dampened when comparing single layer A peak to trough 512 to multilayer group B peak to trough 511 and C peak to trough 513.

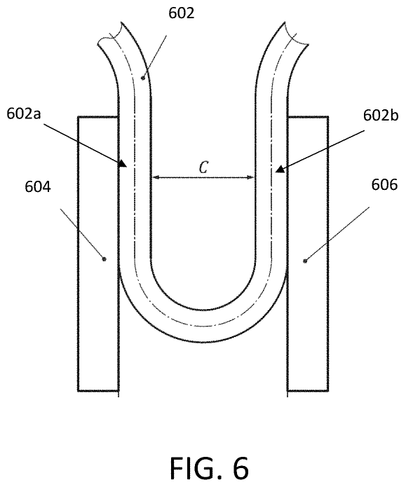

Based on ISO 10619-1_2017 standard, testing was performed on four different types of tubing for measuring flexibility of plastic tubing. The testing setup is shown in FIG. 6. The testing involved folding a piece of tubing 602 in half until a certain gap C is reached between segments 602a and 602b of tubing 602 using a first plate 604 and second plate 606, and measuring the force needed to cause the bend (Fₚₑₐₖ) using a force gauge (not shown) known in the art. It is at this point that the tubing is in a “kinked” state. In the present testing method, the gap was measured as 0.508″. The gap was decided based on the observation that as the tubing is compressed together between the two flat plates, it is fully kinked when the inner diameter was collapsed. To compare the flexibility of the various tubing samples, this gap was kept constant at 0.508 inches. Based on the following measurements, the multilayer tubing design of the present invention has the lowest forces. The more flexible the tubing, the lower the force. The results of the testing are set forth below in Table 1.

TABLE 1

| | Gap Between | | | | | |
|---|---|---|---|---|---|---|
| | the two | | Nominal | Nominal | Nominal | |
| | segments of | Peak Force | OD of the | ID of the | Wall | Wall |
| | the tubing | when Kinked | Tubing | Tubing | Thk. | Profile |
| Present | Gap = 0.508″ | Fₚₑₐₖ = 0.435 lbf | 0.148 | 0.050 | 0.049 | Double |
| Invention | | | | | | Wall |
| Aspiration | | | | | | |
| Tubing | | | | | | |
| OPO73 | Gap = 0.508″ | Fₚₑₐₖ = 0.905 lbf | 0.148 | 0.056 | 0.046 | Single |
| Aspiration | | | | | | Wall |
| Tubing | | | | | | |
| Third Party 1 | Gap = 0.508″ | Fₚₑₐₖ = 1.750 lbf | 0.152 | 0.048 | 0.052 | Single |
| Aspiration | | | | | | Wall |
| Tubing | | | | | | |
| Third Party 2 | Gap = 0.508″ | Fₚₑₐₖ = 0.875 lbf | 0.125 | 0.056 | 0.033 | Single |
| Aspiration | | | | | | Wall |
| Tubing | | | | | | |

In another embodiment of the disclosed invention, the aspiration tubing may comprise more than two layers. For example, the thickness of each layer may vary among the multiple layers across a cross section to achieve the results described herein. Further, the hardness of the material may be adjusted in each of the layers to achieve the results described herein.

In yet another embodiment of the disclosed invention, a multi-layer tubing construction may be applied similarly to an irrigation tubing system of a phacoemulsification surgical system. The disclosed embodiments of FIGS. 2A and 2B are not limited to aspiration tubing, but merely shown as illustrative examples. For example, an irrigation tubing may be comprised of an inner layer and an outer layer, as shown in cross-sectional views of FIGS. 2A and 2B.

Those of skill in the art will appreciate that the herein described apparatuses, engines, devices, systems and methods are susceptible to various modifications and alternative constructions. There is no intention to limit the scope of the invention to the specific constructions described herein. Rather, the herein described systems and methods are intended to cover all modifications, alternative constructions, and equivalents falling within the scope and spirit of the disclosure, any appended claims and any equivalents thereto.

In the foregoing detailed description, it may be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather are to be accorded the widest scope consistent with the principles and novel features disclosed herein.

I claim:

1. A phacoemulsification surgical system, comprising
a phacoemulsification console;
a surgical cassette connected to the console via at least one interface;
a surgical hand piece; and
at least one aspiration tube connected between the surgical hand piece and the surgical cassette, the at least one aspiration tube comprising multiple layers.

2. The surgical system of claim 1, wherein the at least one aspiration tube comprises an inner layer.

3. The surgical system of claim 2, wherein the inner layer of the at least one aspiration tube comprises a stiff material.

4. The surgical system of claim 3, wherein the stiff material of the inner layer of the at least one aspiration tube comprises, at least in part, one selected from the group consisting of a nylon, a polyvinyl chloride (PVC), or a Pebax material.

5. The surgical system of claim 1, wherein the at least one aspiration tube comprises an outer layer.

6. The surgical system of claim 5, wherein the outer layer of the at least one aspiration tube comprises a soft material.

7. The surgical system of claim 6, wherein the soft material of the outer layer of the at least one aspiration tube comprises, at least in part, one selected from the group consisting of a nylon, a polyvinyl chloride (PVC), or a Pebax material.

8. The surgical system of claim 1, wherein the multiple layers include an inner layer and an outer layer.

9. The surgical system of claim 8, wherein the inner layer comprises a stiff material and the outer layer comprises a soft material.

10. The surgical system of claim 8, wherein the outer layer comprises a material softer than a material of the inner layer.

11. A multi-layer tubing construction for improving kink resistance and reducing post occlusion surge and pump pulsations during phacoemulsification surgery, the tubing construction comprising:
an inner layer comprising a stiff material; and
an outer layer comprising a soft material.

12. The multi-layer tubing construction of claim 11, wherein the stiff material comprises, at least in part, one selected from the group consisting of a nylon, a polyvinyl chloride (PVC), or a Pebax material.

13. The multi-layer tubing construction of claim 11, wherein the soft material comprises, at least in part, one selected from the group consisting of a nylon, a polyvinyl chloride (PVC), or a Pebax material.

14. The multi-layer tubing construction of claim 11, wherein the tubing construction is aspiration tubing.

15. The multi-layer tubing construction of claim 11, wherein the tubing construction is irrigation tubing.

16. A phacoemulsification surgical system, comprising
a phacoemulsification console;
a surgical cassette connected to the console via at least one interface;
a surgical hand piece; and
at least one multi-layer tube connected between the surgical hand piece and the surgical cassette.

17. The phacoemulsification surgical system of claim 16, wherein layer of the layers of the at least one multi-layer tube differ in thickness to each layer of the layers of the at least one multi-layer tube.

18. The phacoemulsification surgical system of claim 17, wherein the thickness is distributed between the layers of the at least one multi-layer tube, wherein an inner layer is thicker in relation to an outer layer of the layers.

19. The phacoemulsification surgical system of claim 17, wherein a material hardness is distributed between the layers of the at least one multi-layer tube, wherein an outer layer is softer in relation to an inner layer of the layers.

20. The phacoemulsification surgical system of claim 16, wherein the at least one multi-layer tube is at least one of an aspiration tube or an irrigation tube.
